(19) United States
(12) Patent Application Publication
(10) Pub. No.: US 2003/0143165 A1
(43) Pub. Date: Jul. 31, 2003

(54) NSAID-containing topical formulations that demonstrate chemopreventive activity

(76) Inventors: Allan Evans, Rosslyn Park (AU); Ross McKinnon, Aberfoyle Park (AU)

Correspondence Address: HOGAN & HARTSON LLP, IP GROUP, COLUMBIA SQUARE, 555 THIRTEENTH STREET, N.W., WASHINGTON, DC, 20004, US

(21) Appl. No.: 10/310,824

(22) Filed: Dec. 6, 2002

Related U.S. Application Data
(60) Provisional application No. 60/350,957, filed on Jan. 25, 2002.

(51) Int. Cl. A61K 31/415; A61K 7/42; A61K 31/60; A61K 31/192

(52) U.S. Cl. 424/59; 514/570; 514/165; 514/406

(57) ABSTRACT

Disclosed herein are chemopreventive methods and topical formulations for the prevention and treatment of ultraviolet light-induced skin cancers, pre-cancerous lesions, and hyperproliferative disorders in mammals, such as humans, utilizing doses of non-steroidal anti-inflammatory drugs. Low doses of non-steroidal anti-inflammatory drugs are present in the topical formulations and allow continued regular use over an extended period of time to prevent such disorders. In particular, the present invention is particularly suitable for non-melanoma skin cancers as these cancers tend to appear in areas of the skin that have had excess sun exposure (head, neck and arms) meaning that the chemopreventive agent would not need to be applied over the entire body of the typical patient. Moreover, it is possible to identify “high-risk” individuals within the populations because people who report one episode of NMSC tend to have a high incidence of a subsequent episode.

### CROSS REFERENCE TO RELATED APPLICATIONS

This application claims benefit of priority from the filing date of U.S. provisional patent application Ser. No. 60/350,957, filed Jan. 25, 2002.

### FIELD OF THE INVENTION

The present invention relates to the prevention and treatment of skin cancer and hyperproliferative skin disorders in mammals, including humans, with the use of topical medicaments. More particularly, the present invention relates to the prevention and treatment of non-melanoma skin cancers with topical medicaments that are safe for continued use in target populations.

### BACKGROUND

Skin cancer is one of the most frequently diagnosed cancers among the Western populations. Exposure to ultraviolet light (UV light) is widely acknowledged to be the major etiologic factor in the development of skin cancers such as squamous and basal cell carcinomas, and it is also a risk factor for the development of melanomas. UV light has also been found in various studies to cause inflammation of the skin, epidermal hyperplasia, and changes in the expression of numerous genes associated with cell proliferation and differentiation, eicosanoid and cytokine production, and growth factor synthesis and responsiveness.

An examination of the incidence of non-melanoma skin cancer (“NMSC”), which includes squamous and basal cell carcinomas, indicates that it is a disease associated with significant morbidity and which generally requires surgical intervention and costly medical care.

NMSC is the most common type of cancer for males and females in the white population, with most NMSC occurring in people over 40 years of age. People with light complexions, fair or red hair and who tend to burn easily on exposure to the sun (Fitzpatrick skin grades 1 and 2) are more prone to develop NMSCs than those with dark-skin. Males have been identified to be at higher risk. Recently, studies have suggested that heavy exposure to the sun in the first few decades of life may be of the greatest importance in determining risk.

Incidence rates for NMSC in Australia are believed to be the highest in the world with a causal relationship with excessive exposure to solar ultraviolet radiation. The Australian Bureau of Statistics indicates approximately 270,000 annual diagnoses of NMSC, which equates to approximately 1.5% of the Australian population. It should be noted, however, that not all NMSCs are often reported to cancer registries. Thus, it is likely that the number of actual annual cases of NMSC is significantly higher. The incidence of NMSC in white populations increases proportionally with proximity to the equator. In 1993-94 the direct cost of treatment of non-melanoma skin cancer in Australia was estimated by the NSW Cancer Center at around $232 million - much more than any other cancer.

Likewise, NMSC constitute more than one third of all cancers in the US. Healthcare costs associated with the treatment of skin cancers have been reported to be over 500 million annual in the US.

Notably, 75% of all skin cancers are basal cell carcinomas (“BCC”). Recent population studies in Australia indicate incidence rates of between 1-2% for BCC. For example, the incidence rate in south eastern Queensland in particular for those aged 20-69 years was found to be 2.4%. In South Wales in 1998 the incidence of BCC in individuals over 75 years of age was approximately 5 times higher that that of individuals between 50 and 55 years old indicating an age related relationship.

In 1994, the incidence rate of BCC in America was calculated to be 0.3% with rates increasing at over 10% per year with a lifetime risk of up to 30%. It is currently projected by some investigators that, given current rates, 1 in every 5 Americans will develop a skin cancer of some sort during their lifetime.

Thus, NMSC, and BCC in particular, present a significant health risk throughout the world and generate significant health care costs.

Once a patient is diagnosed as having a BCC, the risk of developing a new BCC is highest in the first year thereafter. A recent analysis by Marcil and Stern, published in Archives of Dermatology in 2000, of the data collected in 7 published studies has established that the 3-year cumulative risk for developing a subsequent BCC after the diagnosis of a first BCC is 44% on average. Thus, a person once diagnosed as having a BCC can be considered to be part of a high-risk population for developing new BCCs. Increases in risk for subsequent development of squamous cell carcinomas was also found. There is also a strong association between the risk of developing a subsequent skin cancer and the number of prior skin tumours. In one study the risk was increased from 38% for patients with fewer than 3 previous NMSCs to 93% for patients with 3 to 9 previous NMSCs. Thus, the more prior skin cancers a patient has had, the higher the risk is that that patient will develop skin cancers in the future.

Although the use of physical and chemical sunscreens plays an important role in protection of humans against exposure to UV light, the high incidence of skin cancer among the population means that additional prevention and treatment strategies need to be developed. In particular, a safe and effective preventive strategy is needed for use by those individuals who are most susceptible to this life-threatening disease.

The use of specific natural or synthetic agents (usually non-cytotoxic) to reverse, suppress or prevent cancer is referred to as “cancer chemoprevention.” An ideal chemopreventive agent should not only be effective, but it should be safe enough to be used in a target population without causing unnecessary or unacceptable toxicity. It is important that the perceived benefit (lower risk of cancer) should be balanced by its safety profile (low risk of adverse events). Agents that have been found in studies to hold promise for cancer chemoprevention include vitamin A and green tea for skin cancer and tamoxifen and raloxifene for breast cancer. One group of drugs that has also been researched as potential chemopreventive agents are the non-steroidal anti-inflammatory drugs (“NSAIDs”).

NSAIDs are a group of structurally diverse compounds used clinically for the successful treatment of a range of disorders that are associated with pain and/or inflammation (including arthritic disorders). NSAIDs are known to inhibit the cyclooxygenase (“COX”) enzymes, which catalyse the conversion of arachidonic acid to the various prostaglandins, and the drugs are believed to exert their analgesic and antiinflammatory effects through inhibition of COX. Two isoforms of the COX enzyme have been identified in eukaryotic cells, COX-1 and COX-2. The COX-1 protein is constitutively expressed (i.e., it is present under normal conditions and does not need to be induced) and is involved in the maintenance of homeostatic conditions. For example, COX-1 plays a role in blood clotting and elicits a protective role in organs such as the gastrointestinal tract. The COX-2 protein, on the other hand, is inducible and is involved in the immediate-early gene response to various stimuli, such as cytokines, growth factors and UV light. Older NSAIDs such as aspirin, ibuprofen and flurbiprofen inhibit both forms of COX and are referred to as non-selective NSAIDs. Newer agents, such as celecoxib and rofexocib, are more selective for COX-2 and are referred to as COX-2 selective agents. The ability of NSAIDs in general to reduce inflammation of the skin is acknowledged and is likely to be due to inhibition of epidermal and dermal prostaglandin production.

Over recent years, several lines of investigation have provided evidence that some NSAIDs might be useful for the chemoprevention of certain forms of cancer. For example, sulindac and flurbiprofen, both of which are a non-selective NSAID, can inhibit certain forms of intestinal and prostate cancer in animal models and possibly humans. Furthermore, a study by Pentland et al. that was published during 1999 in “Carcinogenisis” reported that the COX-2 inhibitor, celecoxib, caused a reduction in ultraviolet light-induced skin cancer in mice when administered orally to those animals. Unfortunately, celecoxib is a COX-2 inhibitor that would likely have unjustifiable toxic side effects in humans if continuously ingested as a chemopreventive therapy for skin cancer.

Furthermore, U.S. Pat. No. 5,639,738 and 6,147,059 to Falk et al. teach the use of topical mixtures containing up to 3% by weight NSAIDs, hyaluronic acid and other excipients for the treatment of various skin disorders, including actinic keratosis and basal cell carcinoma. The topical mixtures disclosed in the Falk patents are meant to be applied several times daily, over a period of 3-4 weeks, directly upon the affected skin region to help break down and clear lesions. These mixtures, however, are not for preventive therapies and would be unsuitable for use as a chemopreventive agent over extended periods in a target population.

Solaraze is the trade name for a product approved for topical use in treating actinic keratosis, and the product is currently marketed by Bioglan Pharma, Inc. Solaraze is a gel solution containing 3% by weight of active ingredient diclofenac sodium, an NSAID, and is directly applied twice daily to the immediate areas of the actinic keratosis lesions. The Solaraze product, while providing dermatologists with a chemical agent to treat actinic keratosis lesions, does not provide an effective chemopreventive agent for skin cancers and other hyperproliferative skin disorders that is also safe for continued use in a patient population.

It is apparent from the background of the art as described above that there is a clear need for an effective chemopreventive agent for NMSC. Currently, no such treatment exists, otherwise the recurrence rate would not be so high. Furthermore, there is a need for a chemopreventive agent that can be safely administered to patient populations in high risk of NMSC. Additionally, there remains a need for chemopreventive agents and medicaments that can be applied directly onto the surface of the skin for prevention and/or treatment of skin cancer and other hyperproliferative skin disorders so as to limit systemic complications of the agent.

### SUMMARY OF THE INVENTION

In light of the above-described and other limitations in the prior art, it is an object of the present invention to provide a chemopreventive agent that is safe for continued use in a target population, but is effective in preventing the occurrence of skin cancer and related lesions.

Additionally, it is an object of the present invention to provide an effective chemopreventive mixture for the prevention and treatment of non-melanoma skin cancers that is safe for regular topical application over large portions of the human body over an extended period of time, such as months or years.

Similarly, it is an object of the present invention to provide preventive therapies for non-melanoma skin cancers and hyperproliferative disorders for use in high-risk patient populations.

Furthermore, it is an object of the present invention to provide a topical chemopreventive formulation which is effective in preventing non-melanoma skin cancers, but which has low toxicity.

Likewise, it is an object of the present invention to provide a topical chemopreventive formulation which is effective in preventing non-melanoma skin cancers, but which causes no appreciable COX-1 and COX-2 inhibition systemically, and preferably also topically.

Also, it is an object of the present invention to provide a chemopreventive product that can be safely prescribed to and used by patients diagnosed with a non-melanoma skin cancer for use on all areas of the body that have a history of significant exposures to ultraviolet light.

To achieve these and other objects of the invention, the present invention stems from the discovery that regular topical doses of non-steroidal anti-inflammatory drugs may be used to prevent and treat ultraviolet light-induced skin cancers, pre-cancerous lesions, and hyperproliferative disorders in mammals, such as humans.

In particular, NMSC is an ideal disease for the utilization of the topical skin cancer preventive methods and formulations according to the present invention because these cancers tend to appear in areas of the skin that have had excess sun exposure (head, neck and arms) meaning that the chemopreventive agent would not need to be applied over the entire body of the typical patient. Moreover, as shown below, it is possible to identify “high-risk” individuals within the populations because people who report one episode of NMSC tend to have a high incidence of a subsequent episode.

One embodiment of the present invention comprises methods for preventing the occurrence of non-melanoma skin cancers in a patient. These methods entail regularly applying a topical formulation to the skin of said patient, where the formulation contains a pharmaceutically effective amount of a non-steroidal anti-inflammatory drug. Preferably, those NSAIDs are arylpropionic acid derivatives and in concentrations of up to approximately 2% w/v.

Additionally, embodiments of the present invention comprise methods for preventing skin disorders in a patient where the skin disorders are related to the hyperproliferation of skin cells. Theses methods include the regular application of a topical formulation to the skin of said patient where the formulation includes a carrier medium containing up to approximately 2% w/v of a NSAID. Preferably, regular application of the topical formulation entails applying the formulation at least once daily to areas of the body that have historically been exposed to ultraviolet light, such as the head neck and arms.

Furthermore, embodiments of the present invention also pertain to topical pharmaceutical formulations containing NSAIDs. One such embodiment includes a carrier medium and a NSAID of the arylpropionic acid derivative class present in a concentration of up to approximately 2% w/v. Another such embodiment has as essential ingredients a non-toxic and pharmaceutically inert carrier medium, and up to approximately 2% w/v of a NSAID.

Particularly suitable NSAIDs for use in embodiments of the present invention include those that are arylpropionic acid derivatives, such as alminoprofen, benoxaprofen, bermoprofen, carprofen, cicloprofen, ketoprofen, fenoprofen, flunoxaprofen, flurbiprofen, ibuprofen, indoprofen, loxoprofen, microprofen, naproxen, pirprofen, pranoprofen, suprofen, tiaprofenic acid and ximoprofen. In particular, ibuprofen and flurbiprofen can be advantageously utilized in embodiments of the present invention because they exhibit high effectiveness and low toxicity. Other NSAIDs of other classes, such as sulindac, can be employed in embodiments of the invention as will be described below.

Preferred embodiments of the present invention relate to methods for and formulations for topical application of flurbiprofen over a range of concentrations up to approximately 2% w/v, but preferably up to approximately 1% w/v and most preferably up to approximately 0.5% w/v. Administration of such a formulation causes a reduction in the incidence (as well as fewer numbers) of papillomas and tumours of skin cancer and other skin disorders associated with ultraviolet light exposure. The protective effects of topical application of flurbiprofen combined with its low toxicity makes such topical formulations according to the present invention a superior chemopreventive agent against ultraviolet-induced skin cancer in humans and useful for treating patients with a predisposition to skin cancer. The general anti-proliferative effects of flurbiprofen as identified herein demonstrates that flurbiprofen, either in its racemic form or as one of the individual isolated enantiomers, or other similar drugs (arylpropionic acid derivatives), would be useful in the prevention or treatment of a range of disorders in which the skin exhibits abnormal proliferation. Such conditions include psoriasis and actinic keratosis in addition to skin cancer.

In embodiments of the present invention, it is preferred that the NSAID employed as a chemopreventive agent in the topical formulations have a sufficiently low toxicity to enable continued daily use over extended periods of time, such as months or even years. In particular, it is preferred that the NSAID employed has low cyclooxegenase inhibition characteristics while still exhibiting high anti-proliferative properties. Certain preferred embodiments of the present invention provide a topical chemopreventive formulation that is effective in preventing non-melanoma skin cancers, but which causes no appreciable COX-1 and COX-2 inhibition by using enantiomeric R-flurbiprofen as the NSAID since only S-flurbiprofen demonstrates appreciably COX-inhibiting activity.

Various preferred embodiments of the invention will now be described in detail with respect to figures and illustrative laboratory experiments.

### BRIEF DESCRIPTION OF THE DRAWINGS

FIG. 1 is a chart that graphically presents laboratory experiment test data regarding the chemopreventive effectiveness of topical flurbiprofen in hairless mice that have been exposed to doses of ultraviolet light over a period of time.

FIG. 2 is a chart that graphically presents laboratory experiment test data regarding the chemopreventive effectiveness of various dose strengths of topical flurbiprofen in hairless mice that have been regularly exposed to doses of ultraviolet light.

FIG. 3a through FIG. 3d are black and white photographs depicting the skin appearance of hairless mice during various stages of regular exposure to ultraviolet light and chemopreventive treatment with flurbiprofen.

FIG. 4 is a chart that graphically presents laboratory experiment test data regarding the relative effect of racemic flurbiprofen to its individual enantiomers on the in vitro proliferation of a cancerous human skin cell line.

### DETAILED DESCRIPTION OF THE PREFERRED EMBODIMENTS

The present invention is supported by findings in various studies and experiments which demonstrate the advantageous effects of a topically applied formulation containing a NSAID on the development of skin cancer and other hyperproliferative skin disorders in mammals, including humans. In particular, studies and experiments as described herein indicate that racemic flurbiprofen, a known non-selective COX inhibitor and widely used oral NSAID, is particularly effective for the prevention of skin cancer.

Certain of the experiments describe hereafter entail the exposure of hairless mice to a combination of UV-A and UV-B light (approximating the solar spectrum) to demonstrate the efficacy of long-term prevention according to the invention. Hairless mice, as used in the experiments detailed herein, have generally been accepted in the art as a good laboratory test model for predicting the therapeutic results of pharmacological treatments upon mammals, including humans.

In a first laboratory experiment performed, female SKH-1 mice (hairless mice) were purchased from the Animal Resource Service of Murdoch University, Western Australia, at approximately 3-4 weeks of age. Upon arrival at the Applicants' laboratory, the mice were housed in climate-controlled quarters (22±1° C. at 50% humidity) with a 12-hour light/dark cycle in yellow fluorescent lighting. All animals were allowed free access to rodent diet and water. The experimental protocol and all procedures were approved by CSIRO Health Sciences and Nutrition Animal Experimental Ethics Committee and followed the Australian Code of Practice for the care and use of animals for scientific purposes. The animals were observed daily during the period of UV light exposure. Individual body weights were determined twice weekly throughout the whole study period of 28 weeks.

Racemic flurbiprofen (“RS-FB”), used as the chemopreventive NSAID in this experiment, was purchased from Sigma Chemical Company (Sydney, Australia). Several topical formulation solutions were prepared by dissolving RS-FB in 70% ethanol (in water) as the carrier medium to achieve final RS-FB concentrations of 0.5, 1.0, 2.0 and 3.0% w/v. The drug solutions were prepared every two weeks throughout the whole study period.

The SKH-1 mice were divided into 5 treatment groups of 30 animals each. Control animals were treated with vehicle only (70% w/v ethanol in water), while the other groups of animals were treated with 0.5%, 1%, 2%, or 3-2% w/v of RS-FB dissolved in 70% ethanol (3-2% indicates that one treatment group of mice was originally treated with 3% w/v of racemic flurbiprofen but suffered from some signs of adverse effects and, as a result, that treatment group received treatment with 2% w/v of racemic flurbiprofen from week 10 of the study). Within each treatment group, the animals were further divided into 2 subgroups of 15 animals. One subgroup was treated with vehicle or RS-FB solution one hour before the UV light exposure. The other subgroup was exposed to the UV light one hour before the application of vehicle or the allocated RS-FB solution. The solutions were applied topically to the dorsal area of the animals, in a volume of approximately 50-125 μl per application, using cotton tips.

Simulated solar irradiation was provided by an array of 6 UV-A tubes (Sylvania F40BL) symmetrically housed around a single UV-B tube (Philips FL 40SE) in a custom-built unit. As the skin of the animals progressively thickened, the time of exposure to reach 1 minimal erythematous dose (MED) was increased from 7 minutes per day to 10 minutes per day, 5 days a week. The integrated UV-B irradiance (280-320 nm) was 2.4×10⁻⁴ W/cm², and the UV-A (320-400 nm) was 1.8×10⁻³ W/cm² as measured with a model IL 1700 spectro-radiometer (International Light, Newburyport, Mass.). During treatment, the mice in each group were placed in separate open plastic cages in the UV housing unit. No mice exhibited any evidence of undue reddening of the skin, blister formation or skin peeling. Treatment in this manner was continued for 28 weeks.

At all concentrations up to and including 2% topical flurbiprofen there were no signs of adverse effects on the animals during the study or evidence of significant pathology upon sacrifice at the termination of the study. As mentioned previously, animals initially treated with 3% flurbiprofen displayed adverse effects (such as lethargy, dehydration, and diarrhoea) and were subsequently treated with 2% solution.

The development of skin cancer was assessed as the visible appearance of papillomas, which subsequently developed, in a subset of cases, to tumours. Weekly papilloma and tumour counts were performed after the appearance of the first papilloma and were continued until the termination of the experiment (week 28). The diameters of papillomas and tumours were measured and their locations noted. Data was recorded representing the number of (a) papillomas, (b) tumours and (c) papillomas plus tumours per animal. While the natural progression is from papilloma to tumour, it will be appreciated by those skilled in the art that, in some cases, the distinction between a papilloma and a tumour could not be made unequivocally. The data was analysed taking this into account. The appearance of the skin lesions was expressed progressively as incidence (percentage of mice bearing at least one papilloma/tumour) and yield (average number of papillomas/tumours per mouse) as well as burden (area of skin affected at the end of study).

Analysis of variance (ANOVA) was used to determine whether RS-FB administration caused a statistically significant change in the appearance and yield of papilloma/tumours in comparison with the control (vehicle group). Comparisons between groups was performed using the Scheffe method. Significance was concluded when P<0.05.

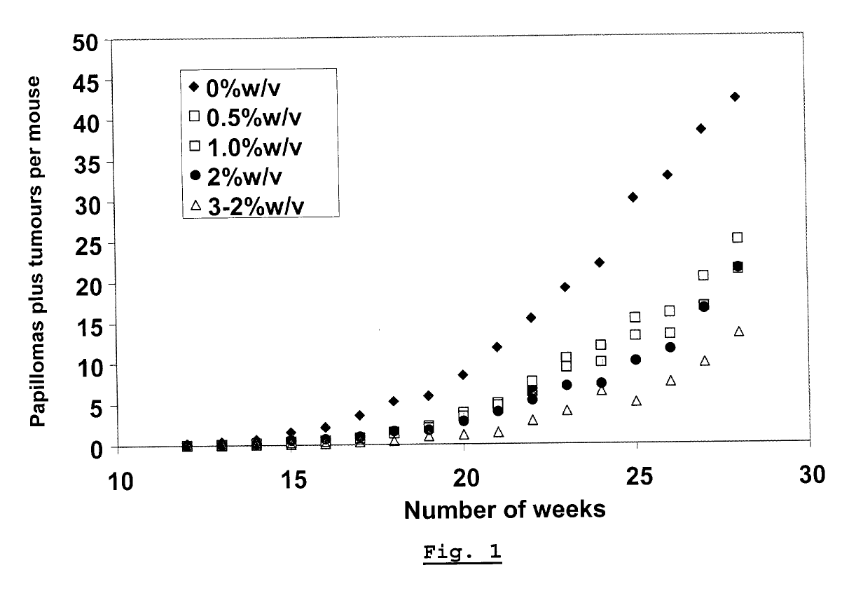

Papilloma and tumour yield data (average number per animal) for the treatment and control groups in this experiment are presented in FIG. 1, which clearly shows the progressive increase in the number of papillomas plus tumours as a function of time (and continued UV exposure). The figure also shows that the papilloma/tumour count steadily increased in the control group compared to all racemic flurbiprofen treatment groups. The differences (between the treatment groups and the control group) became (and remained) statistically significant from week 13 of the study (P<0.05), reaching a level of significance of <0.001. Similar results were obtained when the data were expressed as papillomas only or as tumours only.

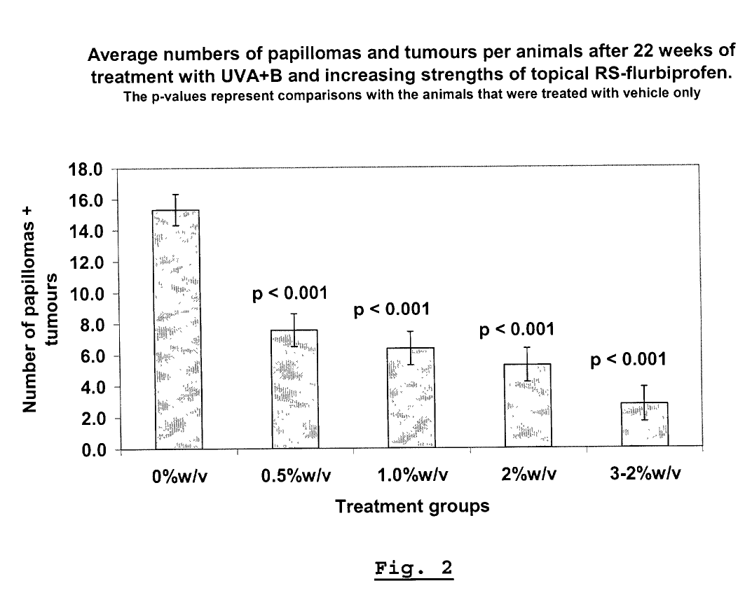

FIG. 2 is a bar graph demonstrating the average number of papillomas plus tumours after 22 weeks of treatment (as a representative description of data from weeks 13 to 28). In all treatment groups, there was a highly significant (p<0.001) reduction in papillomas plus tumours compared with the control group. Similar results were obtained when data from other weeks (week 13 through to 28) were likewise individually analysed statistically.

Although racemic flurbiprofen proved to be effective irrespective of when it was applied in reference to UV-light light exposure (i.e. one hour before or one hour after), by comparing papilloma/tumour incidence between the two subgroups, it appears that topical application of racemic flurbiprofen prior to UV-light provided superior protection against photo-carcinogenesis in the SKH-1 hairless mouse model.

Another method used to evaluate the data from this experiment was to monitor, for each group of mice, the time for 50% of mice within the group to develop at least one papilloma (DP₅₀) or tumour (DT₅₀). For the control group, the DP₅₀ and DT₅₀ values were 14 and 25 weeks, respectively. For the mice treated with 0.5, 1, 2 and 3-2% racemic flurbiprofen, the DP₅₀ values were substantially greater, at 17, 17, 17.5 and 18.5 weeks, respectively. Interestingly, at the end of the study, only 45%, 25%, 25% and 10% of the animals had developed at least one tumour in the 0.5%, 1.0%, 2.0% and 3-2% w/v racemic flurbiprofen treatment groups, respectively. Thus, the efficacy of the topically applied racemic flurbiprofen meant that substantially less animals developed skin tumours within the experimental observation period. Comparing the data from the animals that received the highest concentration of flurbiprofen with the control group, it was found that there was a greater than 80% reduction in the number of animals that developed skin tumours. This result provides clear evidence in support of the benefits associated with topical flurbiprofen administration according to embodiments of the invention.

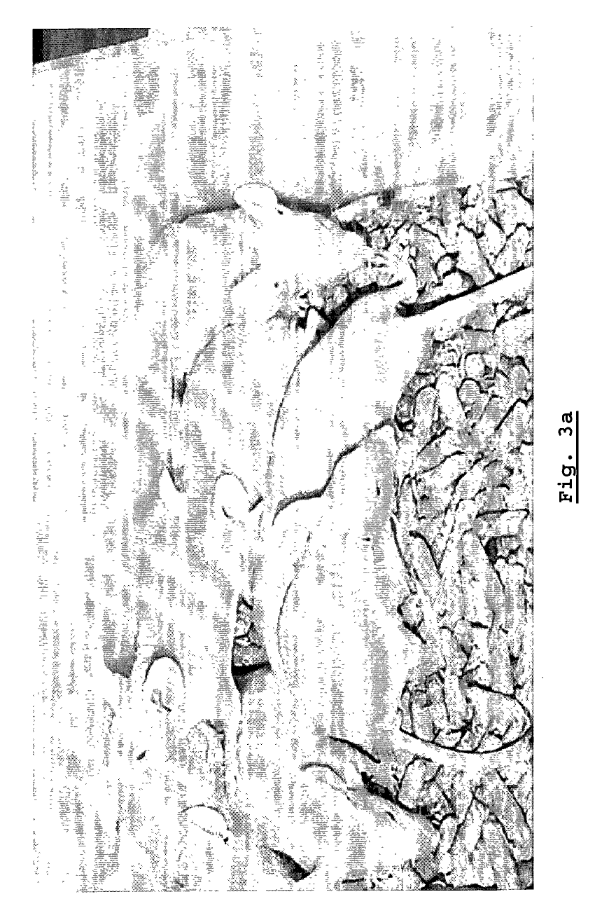

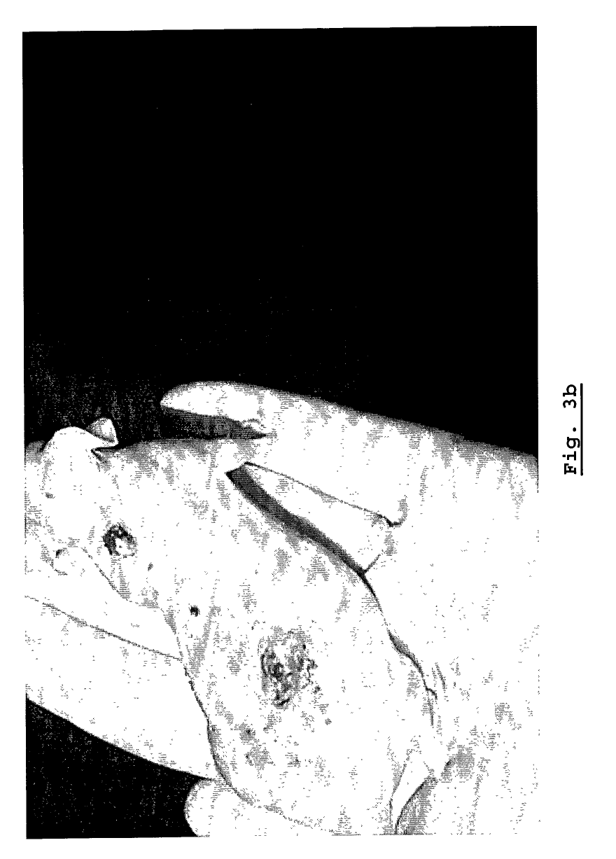

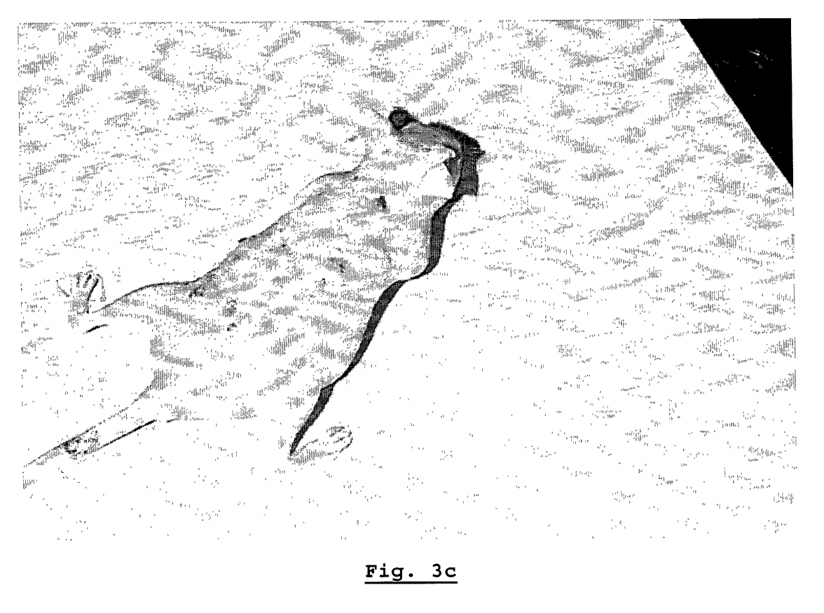

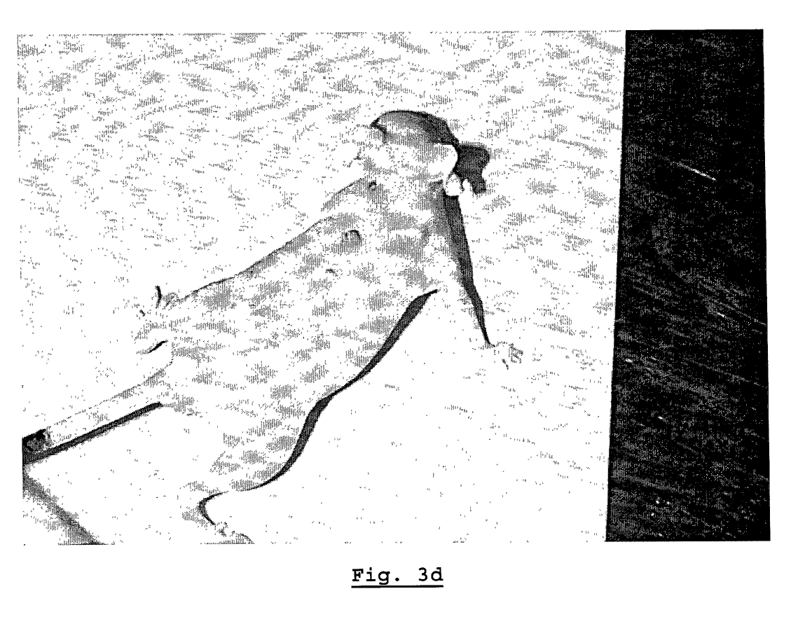

FIG. 3a is a “before” black and white photograph of hairless mice prior to the start of UV light exposure according to the above experiment. FIG. 3b through FIG. 3d are “after” black and white photographs of mice from various groups at the completion of the 28th week. The mouse depicted in FIG. 3b was representative of the control group, having been treated with vehicle only, and displays a high tumour burden with ulcer formation. The remaining two photographs of FIG. 3c and FIG. 3d depict representative animals from the 1% and 2% racemic flurbiprofen treatment groups, respectively. The protection afforded by racemic flurbiprofen is clear in the reduction in tumours and ulcers (compared to the animal in FIG. 3b).

As is apparent from experiments described above with hairless mice and regular topical administration of racemic flurbiprofen, it has been found by the Applicants that the non-selective NSAID exhibits chemopreventive efficacy against skin carcinogenesis in mammals when applied topically as a racemic mixture. Flurbiprofen belongs to the arylpropionic acid derivatives class of NSAIDs that are available in the market extensively as racemates (i.e. they are used as mixtures of two optical isomers, or enantiomers). The flurbiprofen used in the present study was an equal-part mixture of R-flurbiprofen and S-flurbiprofen. Flurbiprofen is approved for use as an analgesic agent and for the treatment of inflammatory conditions.

Further, it should be noted that similar NSAIDs to flurbiprofen, such as those that fall within the arylpropionic acid class of NSAIDs and including, but not limited to, alminoprofen, benoxaprofen, bermoprofen, carprofen, cicloprofen, ketoprofen, fenoprofen, flunoxaprofen, ibuprofen, indoprofen, loxoprofen, microprofen,naproxen, pirprofen, pranoprofen, suprofen, tiaprofenic acid and ximoprofen, are expected to have similar chemopreventive and antiproliferative activity in mammals as both the racemate and as the individual enantiomers, when applied topically.

In experiments described herein, chemical initiators or promoters were not employed since some of these, particularly TPA, are known to cause free radical damage to cells, and thus may not be an appropriate model for UV-induced human skin cancer. Experiments used to substantiate the present invention employed UV irradiation alone. The mixed spectrum that was used closely resembles solar radiation.

In the experiments used to substantiate the present invention, racemic flurbiprofen was found to provide significant protection against the development of skin cancer without oral drug administration. Protection was demonstrated as a reduction in the yield of papillomas and tumours as well as a delay in the onset of tumour development. While not intending to be limited to any particular path of action, it is believed that the excellent results obtained with flurbiprofen may be because the topical administration of racemic flurbiprofen is more effective than the systemic oral dose strategies. This is likely to be due to the delivery of drug, being directly to the site of UV-induced carcinogenesis. In addition, the effects reported previously were likely attenuated, as the dose of oral COX-inhibitor was limited due to side effects such as gastric ulceration.

Importantly, the present invention recognizes that the COX inhibition caused by flurbiprofen is stereoselective, with the (S)-enantiomer capable of inhibiting prostaglandin synthesis via inhibition of COX isoforms, while the R-enantiomer is essentially devoid of COX inhibitory activity. Another experiment, comprising in vitro laboratory tests, provided evidence demonstrating that the individual enantiomers of flurbiprofen are equally potent in terms of their ability to inhibit the proliferation of human skin cancer cells.

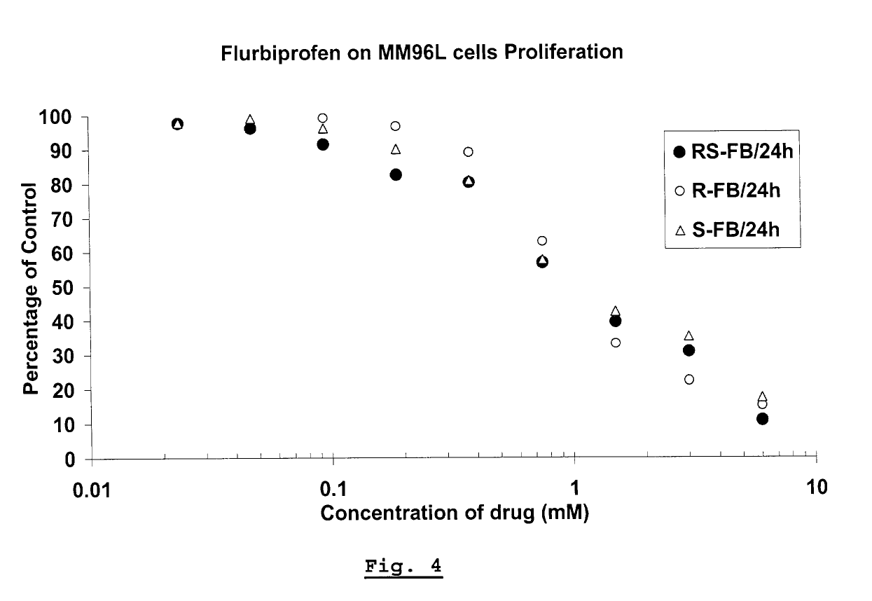

FIG. 4 is a chart that graphically presents laboratory experiment test data regarding the relative effect of racemic flurbiprofen to its individual enantiomers, R-flurbiprofen flurbiprofen (“R-FB”) and S-flurbipofen (“S-FB”), on the in vitro proliferation of a cancerous human skin cell line. For the underlying experiment, the non-pigmented human skin cancer cell line MM96L was studied in vitro utilizing the MTS colorimetric assay to assess cell proliferation. DNA fragmentation assays, acridine orange staining and flow cytometry were also utilized to assess apoptosis and cell cycle effects. As seen by the data depicted in FIG. 4, racemic flurbiprofen and both its enantiomers were all demonstrated to inhibit the proliferation rate and induce apoptosis in both cell lines (data being shown for cells that have been exposed to the treatment for 24 hours). An important finding was that there was no difference between the enantiomers of flurbiprofen in their anti-proliferative potency, despite the fact that only the S-isomer was capable of reducing COX activity.

Therefore, it is to be expected that the chemopreventive effects observed with racemic flurbiprofen would be elicited by both of its enantiomers. This raises the possibility that the chemopreventive effects of topical racemic flurbiprofen are likely to arise from a mechanism that does not involve COX-inhibition. It also raises the possibility of using either enantiomer of flurbiprofen in a topical formulation for the prevention or treatment of skin cancer. Thus, as will be readily appreciated by one of ordinary skill in the art, the use of pure enantiomeric forms of flurbiprofen can be expected to have many potential benefits. Using pure enantiomers will expose the recipient to only the most active isomer, potentially reducing the overall dose required and in doing so, not exposing them to an unnecessary component in the formulation. Adverse effects mediated by the enantiomer (or metabolite(s) of the enantiomer) that is not included in the formulation are also avoided.

As will be understood by one of ordinary skill in the art, one of the most appealing factors for using flurbiprofen in preferred embodiments of the present invention is the potential to possibly separate therapeutic and toxic effects. It is generally accepted in the medical arts that many of the toxic effects associated with NSAID use are due to COX inhibition (particularly with COX-1 inhibition), especially in the gastrointestinal tract and kidneys. Therefore, applying the drug topically will allow high levels of drug at the intended site of action, and comparatively lower levels at significant sites of toxicity. Moreover, using the R-flurbiprofen enantiomer (which exhibits low COX inhibition) will allow the therapeutic effect, if present, to occur without the toxicity of COX inhibition. While it is unclear whether the side effects observed in the hairless mice experiments detailed above, with the highest initial treatment concentration of 3% w/v of racemic flurbiprofen, were due to the inhibition of COX activity, it is reasonable to conclude that it is likely to be the case. Thus, given that the R-enantiomer of flurbiprofen is found to have similar significant positive effects against hyperproliferative activity as does the S-enantiomer, and the fact that R-flurbiprofen has been found to provide chemopreventive activity in some forms of animal intestinal and prostate tumours after oral dosing, it can be concluded that similar results will be obtained in additional photo-carcinogenesis experiments for topical R-flurbiprofen. Topical treatments employing the R-enantiomer of flurbiprofen (that is, the enantiomer non-inhibiting COX activity) would therefore be preferred according to embodiments of the present invention due to the decreased risk in potential toxicity.

The various experiments detailed above have demonstrated that racemic flurbiprofen and its individual enantiomers elicit anti-proliferative (apoptotic) effects when exposed to rapidly dividing human skin cancer cells (see FIG. 4). They also demonstrate that racemic flurbiprofen prevents the development of a proliferative disorder of particular interest (skin cancer) when applied topically to mice exposed to UV light (See FIG. 1 through FIG. 3d). This supports the efficacy of flurbiprofen when used topically to prevent other skin disorders that are characterised by hyperproliferative states. Such disorders include, but are not limited to, hyperkeratosis, psoriasis, actinic keratoses and seborrheic dermatitis.

Further experiments similar to that discussed above with respect to FIG. 4 were conducted to assess the anti-proliferative effects of other NSAIDs on human skin cells. The data collected from these experiments show that aspirin, sulindac and ibuprofen (both the R and S isomers of ibuprofen) exhibited anti-proliferative effects at concentrations that were similar to those found for flurbiprofen. For example, the IC50 values (concentration required to inhibit cell proliferation by 50%) were 1.49 and 1.50 mM for R-and S-ibuprofen, respectively, 0.97 to 5.53 mM for aspirin (depending on the duration of exposures) and 1.03 to 2.16mM for sulindac. These values are similar to the IC50 vales encountered for flurbiprofen.

These results suggest that the anti-proliferative effects of NSAIDs are not limited to the arylpropionic acid class. Indeed, it is likely that the topical anti-proliferative effects that are required for the prevention of skin cancer is a property that will be shared by NSAIDs in general, irrespective of their structural class. This includes the following classes and examples within each class (from Goodman and Gilman's ‘The pharmacological basis of therapeutics’, Ninth Edition, McGraw Hill, New York, page 621, 1996):

1. Salicylic acid derivatives, including aspirin, sodium salicylate, diflunisal, sulfasalazine, olsalazine, aspirin

2. Para-aminophenol derivatives, including paracetamol

3. Indole and indene acetic acids, including indomenthacin, sulindac and etodolac

4. Heteroaryl acetic acids, including diclofenac, tolmetin and ketorolac

5. Arylpropionic acids, including ibuprofen, naproxen, flurbiprofen, ketoprofen, fenoprofen, suprofen, alminoprofen, benoxaprofen, carprofen, cicloprofen, flunoxaprofen, indoprofen, loxoprofen, microprofen, pirprofen, pranoprofen, tiaprofenic acid and ximoprofen

6. Anthranilic acids (fenemates), including mefenamic acid and flufenamic acid

7. Enolic acids, including piroxicam, tenoxicam, phenylbutazone

8. Alkanones, including nabumetone

In the cases where the above compounds exist in multiple isomeric forms (structural or optical) it would be reasonable to assume that all isomeric forms elicit the desired anti-proliferative properties.

Further, the data obtained from these experiments shows that in vitro anti-proliferation of flurbiprofen, aspirin, sulindac and ibuprofen yield IC50 values that are unrelated to those NSAIDs'relative potencies as cyclooxygenase inhibitors. This data is collaborated by the finding that the two enantiomers of flurbiprofen were also equally active in slowing down cell growth despite the fact that only (S)-flurbiprofen can inhibit cyclooxygenase.

Therefore, because cell proliferation effects might be expressed via a mechanism that is independent of cyclooxygenase inhibition, treatment and prevention methods and topical pharmaceutical formulations according to the present invention provide a relatively safe topical therapy for the prevention of skin diseases, such as NMSC, by using those NSAIDs that have the lowest potency in terms of cyclooxygenase inhibition (e.g. ibuprofen, sulindac, flurbiprofen). In this manner, the potential side effects that might be elicited by that fraction of the topical dose that is absorbed into the bloodstream will be minimized.

While in the experiments above, the NSAID was applied in a solution form to the skin using a 70% w/v ethanol solution, it will be readily appreciated by one of ordinary skill in the art that other topical solutions (both with and without alcohols) and formulations, including creams, gels, ointments, oils as well as micro-encapsulations and liposomes, etc., can be used to deliver the active NSAID employed. For example, in preferred embodiments of the present invention, an active NSAID, such as racemic flurbiprofen, can be present at a pharmacologically effective amount in a sun block lotion (i.e., in combination with a UV blocking agent) to be used to prevent onset of UV-induced skin damage and UV-induced skin cancer.

A range of carrier mediums would be suitable for the topical administration of flurbiprofen (or other NSAID or isomer of such) for the prevention of skin cancer. This would include ointments, creams, gels, jellies or other application. The properties of an ideal topical formulation would be one that is easy to apply to a reasonable large area of hairy, and non-hairy skin, requiring the minimum of rubbing and leaving a minimal amount of residue on the surface of the skin. A water-miscible gel containing the active ingredient (alone or in combination) would be just one example of such a formulation. Several examples of possible topical formulations, using 1% w/v flurbiprofen as the NSAID ingredient, are provided below.

### EXAMPLE 1

One suitable water-miscible gel formulation contains the components of table 1 below.

TABLE 1

| | |
|---|---|
| Flurbiprofen | 1% |
| Tragacanth | 2.5% |
| Glycerol | 25% |
| Isopropyl alcohol | 5% |
| Benzyl alcohol | 1% |
| Purified water | to 100% |

To prepare this gel, mix the tragacanth with the glycerol and add most of the purified water. Heat to boiling and allow to cool, mixing during the cooling process. Mix the flurbiprofen in the isopropyl alcohol. Combine the water and alcoholic phases and add benzyl alcohol, and add water to volume. In the above formula, it should be understood that ibuprofen, naproxen or any other NSAID as described herein could be used in place of flurbiprofen. Additionally, it should be understood the concentration of the NSAID could be adjusted within pharmaceutically safe and effective ranges. The percent quantities of one or all ingredient could be adjusted to provide an acceptable product. For topical use, the product would be sterilised using a method that is suitable.

### EXAMPLE 2

Table 2 below provides another suitable gel formulation according to the present invention.

TABLE 2

| | |
|---|---|
| Flurbiprofen | 1% |
| Glycerol | 30% |
| Carbopol 934 | 0.5% |
| Propylene glycol | 2% |
| Benzyl alcohol | 1% |
| Purified water | to 100% |

This gel is prepared in a similar manner to the gel of

### example 1.

EXAMPLE 3

A water-miscible cream such as Aqueous Cream APF would be a suitable emulsion-based formulation to act as a vehicle for flurbiprofen or a related compound. In this example, the formulation provided in table 3 below would apply.

TABLE 3

| | |
|---|---|
| Flurbiprofen | 1% |
| Emulsifying ointment | 30% |
| Glycerol | 5% |
| Phenoxyethanol | 1% |
| Sterile water | to 100% |

In this example, the cream base is prepared by heating the aqueous (glycerol, water, phenoxyethanol) and oil phases (emulsifying ointment) separately to about 60° C., mixing and stirring until cool. The flurbiprofen can be incorporated either by mixing through the oil phase or by levigation with the final cream base.

### EXAMPLE 4

Another suitable formulation is a free-flowing lotion comprising a formulation like that in table 4 below.

TABLE 4

| | |
|---|---|
| Flurbiprofen | 1% |
| Cetomacrogol emulsifying wax | 3% |
| Liquid paraffin | 10% |
| Glycerol | 10% |
| Chlorhexidine gluconate | 0.02% |
| Sterile water | to 100% |

The formulation of table 4 can be prepared by melting the emulsifying wax in the liquid paraffin at 60° C. The water phase containing the glycerol and chlorhexidine is warmed to the same temperature and the two phases are mixed and adjusted to volume with warm water. Again the flurbiprofen can be added by levigation or by incorporation into the oil phase during heating.

The preferred embodiments of the invention being thus described above, it will be readily apparent to one of ordinary skill in the art that many insubstantial changes could be made to the invention without departing from or fundamentally altering the scope of the invention as hereafter claimed.

What is claimed is:

1. A method for preventing the occurrence of non-melanoma skin cancers in a patient, comprising:
regularly applying a topical formulation to the skin of said patient, said formulation containing a pharmaceutically effective amount of a non-steroidal anti-inflammatory drug.

2. The method according to claim 1, wherein said regular application of the drug comprises spreading said topical formulation over skin areas of the patient at least once daily.

3. The method according to claim 2, wherein said skin areas are portions of said patient that have historically been exposed to appreciable ultraviolet light.

4. The method according to claim 3, wherein said portions are selected from the group consisting of the patient's hands, arms, neck, ears, scalp and face.

5. The method according to claim 1, wherein said non-steroidal anti-inflammatory drug is an arylpropionic acid derivative.

6. The method according to claim 5, wherein said non-steroidal anti-inflammatory drug is flurbiprofen.

7. The method according to claim 6, wherein said topical formulation is substantially free of the S-flurbiprofen enantiomer.

8. The method according to claim 5, wherein said non-steroidal anti-inflammatory drug is ibuprofen.

9. The method according to claim 5, wherein said topical formulation comprises said non-steroidal anti-inflammatory drug is present in a carrier medium, and wherein said non-steroidal anti-inflammatory drug is present in a concentration of up to approximately 2% w/v.

10. The method according to claim 9, wherein said non-steroidal anti-inflammatory drug is present in a concentration of up to approximately 1% w/v.

11. The method according to claim 10, wherein said non-steroidal anti-inflammatory drug is present in a concentration of up to approximately 0.5% w/v.

12. The method according to claim 1, wherein said non-melanoma skin cancers comprise basal cell carcinomas, and wherein said patient has been previously diagnosed as having had a basal cell carcinoma.

13. The method according to claim 1, wherein said non-steroidal anti-inflammatory drug is sulindac.

14. The method according to claim 1, wherein said topical formulation consists essentially of said non-steroidal anti-inflammatory drug present in a carrier medium, and wherein said carrier medium is substantially safe for repeated daily use over large portions of the skin.

15. The method according to claim 1, further comprising before said applying, identifying said patient as being at elevated risk of developing a non-melanoma skin cancer.

16. The method according to claim 15, wherein said identifying said patient as being at elevated risk occurs whenever said patient has been diagnosed as having a non-melanoma skin cancer.

17. The method according to claim 1, wherein said topical formulation comprises said non-steroidal anti-inflammatory drug is present in a carrier medium, and wherein said non-steroidal anti-inflammatory drug is present in a concentration of up to approximately 2% w/v.

18. The method according to claim 17, wherein said non-steroidal anti-inflammatory drug is present in a concentration of up to approximately 1% w/v.

19. The method according to claim 18, wherein said non-steroidal anti-inflammatory drug is present in a concentration of up to approximately 0.5% w/v.

20. A pharmaceutical formulation adapted for topical administration upon the skin, comprising:
a carrier medium, and
a non-steroidal anti-inflammatory drug of the arylpropionic acid derivative class present in a concentration of up to approximately 2% w/v.

21. The pharmaceutical formulation according to claim 20, wherein said non-steroidal anti-inflammatory drug of the arylpropionic acid class is present in a concentration of up to approximately 1% w/v.

22. The pharmaceutical formulation according to claim 20, wherein said non-steroidal anti-inflammatory drug of the arylpropionic acid class is present in a concentration of up to approximately 0.5% w/v.

23. The pharmaceutical formulation according to claim 20, wherein said arylpropionic acid class non-steroidal anti-inflammatory drug is flurbiprofen.

24. The pharmaceutical formulation according to claim 23, wherein said flurbiprofen is present in a concentration of up to approximately 1% w/v.

25. The pharmaceutical formulation according to claim 23, wherein said flurbiprofen is present in a concentration of up to approximately 0.5% w/v.

26. The pharmaceutical formulation according to claim 23, wherein said arylpropionic non-steroidal anti-inflammatory drug is substantially purified R-flurbiprofen enantiomer.

27. The pharmaceutical formulation according to claim 26, wherein said S-flurbiprofen is present in a concentration of up to approximately 1% w/v.

28. The pharmaceutical formulation according to claim 26, wherein said S-flurbiprofen is present in a concentration of up to approximately 0.5% w/v.

29. The pharmaceutical formulation according to claim 20, wherein said arylpropionic acid class non-steroidal anti-inflammatory drug is ibuprofen.

30. The pharmaceutical formulation according to claim 29, wherein said ibuprofen is present in a concentration of up to approximately 1% w/v.

31. The pharmaceutical formulation according to claim 29, wherein said ibuprofen is present in a concentration of up to approximately 0.5% w/v.

32. The pharmaceutical formulation according to claim 20, wherein said non-steroidal anti-inflammatory drug contains low cyclooxygenase inhibition activity.

33. The pharmaceutical formulation according to claim 20, wherein said carrier medium comprises a water miscible gel.

34. The pharmaceutical formulation according to claim 20, wherein said carrier medium comprises a water miscible cream.

35. The pharmaceutical formulation according to claim 20, wherein said carrier medium comprises a free-flowing lotion.

36. The pharmaceutical formulation according to claim 20, wherein said carrier medium contains a sunscreen agent.

37. The pharmaceutical formulation according to claim 20, wherein said carrier medium is substantially safe for daily use over large portions of the skin for a period of months.

38. The pharmaceutical formulation according to claim 20, wherein said carrier medium comprises a water miscible gel, and wherein said non-steroidal anti-inflammatory drug comprises flurbiprofen.

39. The pharmaceutical formulation according to claim 20, wherein said carrier medium comprises a water miscible gel, and wherein said non-steroidal anti-inflammatory drug comprises R-flurbiprofen which is substantially free of S-flurbiprofen.

40. A pharmaceutical formulation adapted for topical administration upon the skin, consisting essentially of:
a non-toxic and pharmaceutically inert carrier medium, and
up to approximately 2% w/v of a non-steroidal anti-inflammatory drug.

41. The pharmaceutical formulation according to claim 40, wherein said non-steroidal anti-inflammatory drug is present in a concentration of below approximately 1% w/v.

42. The pharmaceutical formulation according to claim 40, wherein said non-steroidal anti-inflammatory drug is present in a concentration of below approximately 0.5% w/v.

43. The pharmaceutical formulation according to claim 40, wherein said non-steroidal anti-inflammatory drug is flurbiprofen.

44. The pharmaceutical formulation according to claim 43, wherein said flurbiprofen is present in a concentration of below approximately 1% w/v.

45. The pharmaceutical formulation according to claim 43, wherein said flurbiprofen is present in a concentration of below approximately 0.5% w/v.

46. The pharmaceutical formulation according to claim 43, wherein said non-steroidal anti-inflammatory drug is substantially purified R-flurbiprofen enantiomer.

47. The pharmaceutical formulation according to claim 46, wherein said S-flurbiprofen is present in a concentration of below approximately 1% w/v.

48. The pharmaceutical formulation according to claim 46, wherein said S-flurbiprofen is present in a concentration of below approximately 0.5% w/v.

49. The pharmaceutical formulation according to claim 40, wherein said non-steroidal anti-inflammatory drug is ibuprofen.

50. The pharmaceutical formulation according to claim 49, wherein said ibuprofen is present in a concentration of below approximately 1% w/v.

51. The pharmaceutical formulation according to claim 49, wherein said ibuprofen is present in a concentration of below approximately 0.5% w/v.

52. The pharmaceutical formulation according to claim 40, wherein said non-steroidal anti-inflammatory drug is sulindac.

53. The pharmaceutical formulation according to claim 52, wherein said sulindac is present in a concentration of below approximately 1% w/v.

54. The pharmaceutical formulation according to claim 52, wherein said sulindac is present in a concentration of below approximately 0.5% w/v.

55. The pharmaceutical formulation according to claim 40, wherein said carrier medium comprises a water miscible gel.

56. The pharmaceutical formulation according to claim 40, wherein said carrier medium comprises a water miscible cream.

57. The pharmaceutical formulation according to claim 40, wherein said carrier medium comprises a free-flowing lotion.

58. The pharmaceutical formulation according to claim 40, further consisting essentially of an effective amount of a sunscreen agent.

59. The pharmaceutical formulation according to claim 40, wherein said carrier medium is substantially safe for daily use over large portions of the skin for a period of months.

60. A method for preventing skin disorders in a patient where the skin disorders are related to the hyperproliferation of skin cells, said method comprising:
regularly applying a topical formulation to the skin of said patient, said formulation comprising a carrier medium containing up to approximately 2% w/v of a non-steroidal anti-inflammatory drug.

61. The method according to claim 60, wherein said topical formulation is applied to targeted areas of said patient skin at least once daily.

62. The method according to claim 60, wherein said topical carrier medium is safe for continued daily application to said skin over a period of at least one month.

63. The method according to claim 60, wherein said non-steroidal anti-inflammatory drug is of the arylpropionic acid derivative class.

64. The method according to claim 60, wherein said non-steroidal anti-inflammatory drug is flurbiprofen.

65. The method according to claim 64, wherein said topical formulation is substantially free of S-flurbiprofen.

66. The method according to claim 60, wherein said non-steroidal anti-inflammatory drug contains low cyclooxygenase inhibition activity.

67. The method according to claim 60, wherein said non-steroidal anti-inflammatory drug is present in a concentration of up to approximately 1% w/v.

68. The method according to claim 67, wherein said non-steroidal anti-inflammatory drug is present in a concentration of up to approximately 0.5% w/v.
